(19) United States
(12) Patent Application Publication
(10) Pub. No.: US 2012/0251613 A1
(43) Pub. Date: Oct. 4, 2012

(54) METHOD FOR TREATING VITILIGO WITH A PROSTAGLANDIN ANALOGUE

(75) Inventors: Rajeev JAIN, Patiala (IN); Tarun Narang, Chandigarh (IN); Satya Srinivas Chetlapalli, Bangalore (IN)

(73) Assignee: AGILA SPECIALITIES PVT. LTD., Bangalore (IN)

(21) Appl. No.: 13/194,458

(22) Filed: Jul. 29, 2011

(30) Foreign Application Priority Data
Mar. 29, 2011 (IN) 861/DEL/2011
Jun. 9, 2011 (IN) 1639/DEL/2011

(51) Int. Cl. A61K 9/127; A61K 38/44; A61K 31/436; A61Q 19/00; A61K 31/366; A61K 38/03; A61P 17/00; A61K 31/165; A61K 31/56

(52) U.S. Cl. 424/450; 514/622; 424/94.4; 514/291; 514/171; 514/455; 514/18.6

(57) ABSTRACT

A method of stimulating melanogenesis in a skin surface of a patient, by treating the skin surface with a topical formulation comprising an effective amount of 17-phenyl-18,19,20-trinor-PGF2α ethyl amide in a dermatologically acceptable carrier. The topical formulation may be a cream, a gel, a lotion, a spray, an ointment, an aqueous solution, a nonaqueous solution, or a transdermal patch. The dermatologically acceptable carrier may contain an oily carrier or an aqueous carrier.

### BACKGROUND OF THE INVENTION

1. Field of the Invention

This invention relates generally to treatment of vitiligo using a topical formulation containing a prostaglandin analogue. More particularly, this invention relates to treatment of vitiligo using topical bimatoprost.

2. Description of Related Art

In normal skin, varying shades of brown color are seen. This color is the pigment melanin, produced by a cell type known as a melanocyte. Specifically, ultraviolet radiation stimulates melanocytes to proliferate and produce more melanin in a process called melanogenesis. Melanogenesis relates to the reproduction of melanocytes and the generation of granules of the melanin pigment. Normal skin responds to ultraviolet light with an increase in the brown pigment melanin (tanning).

An absence of melanocytes results in an absence of melanin pigment, and thus the melanin-free area is white. Melanocytes are specialized cells, which synthesize melanin by means of specific organelles, the melanosomes. Medical disorders which cause a loss of these melanocytes can result in diseases, such as vitiligo, marked by a loss of skin pigmentation.

Vitiligo is a disfiguring disease which causes depigmentation of the skin. Vitiligo is marked by white lesions on the skin which exhibit an absence of melanocytes and therefore, an absence of melanin. In short, vitiligo causes a destruction of melanocytes. Specifically, melanocytes from patients with vitiligo frequently undergo an immune reaction that induces melanocytes to undergo apoptosis, causing depigmentation of the skin. Vitiligo does not generally cause uniform skin depigmentation; rather, it causes localized depigmentation of specific regions of the skin, while surrounding skin maintains its normal pigment. The depigmented regions of skin, known as vitiliginous lesions, are highly disfiguring, particularly on skin surfaces not normally covered by clothes, i.e., the face, neck, and hands.

Vitiligo affects 1%-2% of the world population, and results from a lack of melanin in the epidermis due to the disappearance of melanocytes from the skin. The disease is characterized by the appearance of white patches, mainly located on the face and limbs, disregarding the patient's age, sex or geographic location. Frequently, these white patches have a symmetrical distribution on both halves of the body. The result is cosmetically disfiguring, especially for dark skinned people. Furthermore, the depigmented skin is sun sensitive, and thus is subject to sunburns and skin cancer.

There are several types of vitiligo. Generalized vitiligo, or vitiligo vulgaris, is frequently subject to spreading over greater areas of the skin. Generalized vitiligo is marked by highly symmetrical patterns of depigmentation in the skin. Acral or acrofacial vitiligo and localized vitiligo, frequently referred to collectively as focal vitiligo, generally does not spread. Segmental vitiligo, in which depigmentation is generally restricted to one side of the body, also does not typically spread. Focal and segmental vitiligo are largely restricted to isolated patches of skin, and exhibit asymmetrical distribution of depigmented patches of skin. Finally, mucosal vitiligo causes depigmentation of the mucous membranes of the mouth and genitals.

Vitiligo can begin at any age and become gradually progressive to the point of affecting the entire skin. Although the precise cause of vitiligo is not known, such clinical behavior suggests an internal or systemic etiology. It is possible that vitiligo is an autoimmune condition. Studies have reported that patients with vitiligo exhibit a circulating autoantibody that binds to melanocytes in human skin, nevus cells and melanoma cells.

Despite extensive therapeutic efforts over the years, the treatments available are unsatisfactory. Various solutions have been proposed, including:

applying dyes that are intended to color the skin and/or hair to match their natural color;

stimulating the natural pigmentation pathways; or

depigmenting unaffected skin to match vitiliginous areas.

While some of these methods are effective to a greater or lesser degree, most have drawbacks. One method of treating vitiligo involves the use of pseudocatalase cream (PCAT), which should be applied twice daily. PCAT contains calcium chloride, manganese chloride, sodium bicarbonate, and distilled water. PCAT inhibits the progression of pigment loss, and reduces the increased levels of peroxides which are found in the skin of vitiligo patients. However, there are certain drawbacks to PCAT, including causing pimples and ingrown hairs. Also, patients should be screened for phenylalanine deficiency first, as PCAT is more effective for people with phenylalanine deficiency.

Sinvitil, or dermabest gel, which works as a tanning accelerator, has also been tried as a vitiligo treatment. This is a formulated gel containing oils, distilled water, glycerin, carboxmethylcellulose, camphor, menthol, and kathon (a preservative), which is used in conjunction with exposure to the sun or a sun lamp for it to work. Novitil, an enhanced version of Sinvital including polypeptides, Aloe Barbadensis, and oligoelements, has also shown promise in repigmentation therapy. However, these gels must be left on for at least two hours after treatment, significantly restricting the patient's mobility.

Another topical treatment is V-Tar, a 30% standardized water soluble coal tar product which also contains natural anti-inflammatory agents, skin conditioners, and antioxidants. V-Tar has been used successfully in many patients with vitiligo and other hypopigmentary disorders. It will not stain the skin, and its once weekly application is convenient for many patients. Although frequently effective, many people have been found to have an allergic reaction to aromatic compounds in V-Tar.

Other topical treatments include the use of steroid creams or immunosuppressant drugs. In theory, topical immunosuppressant drugs, i.e., tacrolimus (Protopic), reduce the immune response to the skin, curbing the immune response in vitiliginous skin. However, both steroids and topical immunosuppressant drugs can cause significant unwanted side effects. Application of steroid creams to white patches of vitiliginous skin for three months can aid in skin repigmentation; however, this treatment can cause thinning of the skin, blood vessel formation, steroid induced acne, atrophy, joint problems, and possibly arthritis. Those who have tried topical tacrolimus report burning and itching as side effects.

PUVA, a phototherapy treatment involving use of psoralen, in conjunction with ultra violet A (UVA) light is another common treatment. In one frequently-used regimen, oral doses of psoralen are taken two hours before exposure to UVA light or to sun light. Normally, the patient wears wraparound sunglasses to protect his or her eyes. Alternatively, topical psoralen therapy, in which psoralen is applied to skin 30 minutes before exposure to UVA light until white, vitiliginous skin turns pink, may be used. These treatments, however, have significant side effects, including sunburn, redness, blistering, and inflammation. PUVA treatments are not recommended for children under age 10 because phototherapy can cause eye damage and cataracts. Oral psoralen can also cause severe stomach upset, gastrointestinal upset, nausea, and liver toxicity. Phototherapy with psoralen has also been linked to some forms of cancer, including nonmelanoma skin cancer. In the case of oral psoralen, the patient must avoid sunlight for 12-24 hours after treatment. The patient must wear special sunglasses for two hours following treatment. Maintenance treatments are required to see continued improvement. Also, psoralen is capable of causing dermatitis and necrosis of the skin after topical application.

Narrow band UVB or excimer laser treatment of vitiliginous skin consists of highly concentrated beams of narrow band UV light. This procedure maximizes delivery of narrow band UVB radiation to the tissue requiring treatment, while minimizing exposure to superfluous UV radiation. However, this form of phototherapy can also cause burning, like PUVA. This sunburn is less severe than other forms of UV light, but it is not safe around the eyes. Some patients have, however, reported significant success.

In particularly severe cases of vitiligo, where the patches of white skin cover large parts of the body surface, depigmentation is sometimes used. In this therapy, the drug monobenzylether of hydroquinone (monobenzone or Benoquin) is applied to skin twice a day until pigmented skin fades until it matches the patches of depigmented skin. The patient must avoid skin to skin contact with other people for at least 2 hours after application of the drug. Some patients have allergies to Benoquin, and show contact dermatitis, including inflammation (redness and swelling), rash, itching, and dry skin. The rash, inflammation, and itching have been likened to poison ivy. Depigmentation once completed, leaves the patient extremely sensitive to sunlight. This treatment is, in most cases, permanent.

If desired by the patient, surgical procedures may be explored. These include skin grafting and melanocyte transplant. In skin grafting, pieces of normal skin are removed and placed in white skin patches. Melanocyte transplant involves placement of a sample of normally pigmented skin in a laboratory dish with a solution to grow melanocytes. After the melanocytes have multiplied, they are transplanted into the depigmented skin or depigmented skin of the depigmented hair. Both of these procedures are invasive, and may result in infections, scarring, and/or uneven skin pigmentation.

As described above, there is a long-felt need for safe, effective, and non-invasive treatments for vitiligo and other disorders related to skin depigmentation.

### SUMMARY OF THE INVENTION

In light of the present need for improved treatments for vitiligo, a brief summary of various embodiments is presented. Some simplifications and omissions may be made in the following summary, which is intended to highlight and introduce some aspects of the various embodiments, but not to limit the scope of the invention. Detailed descriptions of a preferred embodiment adequate to allow those of ordinary skill in the art to make and use the inventive concepts will follow in later sections.

Various embodiments disclosed herein relate to a method of stimulating melanogenesis in a skin surface of a patient, by treating the skin surface with a topical formulation comprising an effective amount of 7-[3,5-dihydroxy-2-(3-hydroxy-5-phenyl-pent-1-enyl)-cyclopentyl]-N-ethyl-hept-5-enamide (17-phenyl-18,19,20-trinor-PGF2α ethyl amide, or bimatoprost) in a dermatologically acceptable carrier. The topical formulation may be a cream, a gel, a lotion, a spray, an ointment, an aqueous solution, a nonaqueous solution, or a transdermal patch. The dermatologically acceptable carrier may be an oily carrier or an aqueous carrier.

In various embodiments, the carrier comprises water in combination with an excipient, such as benzalkonium chloride, sodium chloride, sodium phosphate dibasic, citric acid, or a mixture thereof. The carrier may further comprise liposomes. The aqueous carrier preferably has a pH of between about 4.0 and 10.0, more preferably between about 6.8 and about 7.8.

The carrier may comprise a buffer to control pH. The buffer may comprise at least one of NaOH and HCl.

In various embodiments, the topical formulation of bimatoprost is used to treat a patient suffering from vitiligo in any of its forms. More particularly, the topical formulation of bimatoprost is used to treat a patient suffering from a condition selected from generalized vitiligo, acrofacial vitiligo, localized vitiligo, and segmental vitiligo. Once repigmentation is achieved, patients having acrofacial vitiligo, localized vitiligo, or segmental vitiligo may stop treatment with topical bimatoprost with little risk of relapse. Patients suffering from generalized vitiligo have greater risk of relapse if treatment with topical bimatoprost is stopped.

Accordingly, various embodiments disclosed herein relate to a method of treating a patient suffering from generalized vitiligo, by treating a depigmented skin surface of the patient with a topical formulation comprising first dosage of bimatoprost in a dermatologically acceptable carrier, wherein the first dosage promotes repigmentation. The treatment is carried out at regular intervals until repigmentation of said depigmented skin surface is complete. The method of treating a patient suffering from generalized vitiligo may comprise a second step of treating the formerly depigmented skin surface of the patient with a topical formulation comprising a maintenance dosage of bimatoprost in a dermatologically acceptable carrier, where the maintenance dosage is less than the first dosage and serves to prevent loss of pigment, or relapse. The reduced maintenance dosage may be achieved by reducing the concentration of bimatoprost in the topical formulation, or by reducing the frequency at which topical bimatoprost is administered.

### DETAILED DESCRIPTION OF THE PREFERRED EMBODIMENTS OF THE INVENTION

According to various embodiments disclosed herein, depigmented skin is treated with a topical composition comprising 17-phenyl-18,19,20-trinor-PGF2α ethyl amide (bimatoprost) to stimulate melanogenesis. The active principle bimatoprost stimulates the synthesis of melanin pigment in the skin and the release of melanosomes from melanocytes of surrounding skin, without causing serious side effects.

Various embodiments relate to a method of stimulating pigmentation in a depigmented skin region of a patient, where the depigmented skin region is surrounded by or adjacent to a nondepigmented skin region. A depigmented skin region is here defined as a skin region having fewer melanocytes per square centimeter than the surrounding or adjacent nondepigmented skin region. The method includes a step of treating the depigmented skin region with a topical formulation comprising an effective amount of bimatoprost in a dermatologically acceptable carrier. In various embodiments, the depigmented skin region has no melanocytes.

Bimatoprost is used topically in eye drops to control the progression of glaucoma and in the management of ocular hypertension. Bimatoprost reduces intraocular pressure by increasing the outflow of aqueous fluid from the eyes. Bimatoprost has also been approved by US FDA for cosmetic use to lengthen eyelashes. The prior art does not teach use of bimatoprost in the treatment of localized skin depigmentation, i.e., vitiligo.

In various embodiments, a topical composition comprising bimatoprost and a dermatologically acceptable vehicle is used for the treatment of vitiligo. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). Bimatoprost is only slightly soluble in water; therefore, in such emulsions, bimatoprost normally is present in the oil phase. In some embodiments, an oil phase containing bimatoprost is emulsified in an aqueous gel. In some embodiments, bimatoprost is dissolved or dispersed in an oil or wax carrier.

In various embodiments, bimatoprost may be topically administered using a transdermal patch. In various embodiments, the transdermal patch comprises an adhesive layer for adhering the patch to the skin, and a drug-impermeable backing layer. In some embodiments, the adhesive layer contains the drug bimatoprost in combination with an adhesive polymer. In this type of system, bimatoprost is released from the adhesive layer and passes directly to the skin.

In other embodiments, the transdermal system has a reservoir layer containing the drug bimatoprost. The drug reservoir layer is a liquid, gel, or semisolid compartment containing a drug solution or suspension, where the reservoir layer is positioned in between the adhesive layer and the backing layer. In this type of system, bimatoprost is released from the reservoir layer and passes through the adhesive layer.

Transdermal patches are useful primarily when vitiliginous patches or lesions on the skin are small. Transdermal patches may also be made with UV-opaque backings, which help to prevent burning of depigmented skin from exposure to the sun.

The pharmaceutical excipients used in the topical preparation of the invention may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic topical bimatoprost formulation include water; ethyl alcohol; isopropyl alcohol; mixtures of water and ethyl and/or isopropyl alcohols; glycerin; ethylene, propylene or butylene glycols; DMSO; and mixtures thereof. Suitable solvents for a hydrophobic topical bimatoprost formulation include mineral oils, vegetable oils, and silicone oils. If desired, bimatoprost may be dissolved or dispersed in a hydrophobic oil phase, and the oil phase may then be emulsified in an aqueous phase comprising water, alone or in combination with lower alcohols, glycerin, and/or glycols.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the topical bimatoprost formulations include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof, including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the topical formulations. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polydydric alcohol esters may be used as emulsifiers or emollients. Suitable polydydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in topical bimatoprost formulations include anionic, cationic, nonionic, and zwitterionic surfactants. Preferred ionic emulsifiers include phospholipids, such as lecithin and derivatives.

Lecithin and other phospholipids may be used to prepare liposomes containing bimatoprost. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals such as bimatoprost can be dissolved into the phospholipid bilayer membrane. The lipid bilayers of the liposomes deliver bimatoprost to melanocytes by fusing with the cell membrane of the melanocytes.

In various embodiments, the topical formulation may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent. The bimatoprost is dissolved in the oil droplets.

Sterols including, for example, cholesterol and cholesterol fatty acid esters; amides such as fatty acid amides, ethoxylated fatty acid amides, and fatty acid alkanolamides may also be used as emollients and/or penetration enhancers.

The topical bimatoprost formulations may also include hydrophilic solvents, including water, lower alcohols, i.e., ethanol or isopropanol, DMSO, DMA and ethers. Bimatoprost has a very low solubility in water. To increase bimatoprost solubility, water may be mixed with hydrophilic organic solvents, i.e., ethanol, isopropanol, or DMSO.

Suitable viscosity enhancers or thickeners which may be used to prepare a viscous gel or cream with an aqueous base include sodium polyacrylate, xanthan gum, polyvinyl pyrollidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols.

Suitable preservatives and/or antioxidants for use in topical bimatoprost formulations include benzalkonium chloride, benzyl alcohol, phenol, urea, parabens, butylated hydroxytoluene (BHT), butylated hydroxyanisole (BHA), Tocopherol, and mixtures thereof.

Suitable chelating agents for use in topical bimatoprost formulations include ethylene diamine tetraacetic acid, alkali metal salts thereof, alkaline earth metal salts thereof, ammonium salts thereof, and tetraalkyl ammonium salts thereof.

In various embodiments, bimatoprost may be dissolved or dispersed in a suitable oily or non-oily liquid or gel vehicle, optionally incorporating absorption enhancing ingredients such as liposomes, for selective application on specific sensitive sites. The liquid or gel vehicle may be contained within a gelatin-based shell made from a combination of gelatin, water, and a plasticizer such as glycerin and/or sorbitol(s). The shell may then be cut or broken to release the liquid or gel vehicle for application to depigmented skin.

The carrier for bimatoprost preferably has a pH of between about 4.0 and 10.0, more preferably between about 6.8 and about 7.8. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid.

The various embodiments of creams, ointments, lotions, solutions, gels, sprays and patches may incorporate bimatoprost as the active ingredient, in combination with penetration enhancing agents and other active agents acting synergistically on the skin for the treatment of vitiligo. In various embodiments, bimatoprost may be incorporated into pseudocatalase cream, dermabest gel, or V-Tar to enhance the effectiveness of these formulations against vitiligo. Other suitable active agents which may be combined with bimatoprost include tacrolimus, pimecrolimus, steroids, psoralen, methoxsalen, trioxsalen, glatiramer acetate, and topical calcineurin inhibitors. Tacrolimus and/or pimecrolimus may be included in an amount of about 1% by weight. If desired, topical bimatoprost may be combined with UV treatments to stimulate repigmentation.

Various embodiments of the bimatoprost formulation include bimatoprost in a concentration of from about 0.005% by weight to about 50% by weight. Formulations containing bimatoprost in an aqueous carrier typically contain from about 0.005% by weight to about 0.5% by weight bimatoprost, preferably about 0.01% by weight to about 0.1% by weight, more preferably about 0.01% by weight to about 0.05% by weight. Formulations containing bimatoprost in an oil or wax carrier typically contain from about 0.005% to about 50% by weight of bimatoprost, preferably about 0.01% by weight to about 25% by weight, more preferably about 0.1% by weight to about 10% by weight, most preferably from about 0.1% by weight to about 5% by weight. Creams, lotions, or other emulsions containing an oil phase and an aqueous phase typically contain bimatoprost in an amount of from about 0.005% to about 25% by weight, preferably about 0.005% to about 10% by weight, more preferably about 0.01% to about 5% by weight. The creams, lotions, or other emulsions may be prepared as water-in-oil or oil-in-water emulsions; in either case, the hydrophobic compound bimatoprost is dissolved or dispersed in the oil phase.

A suitable formulation comprises bimatoprost in a concentration of from about 0.1 to about 0.3 mg/mL (0.01% to 0.03%), and the preservative benzalkonium chloride in a concentration of from about 0.05 to about 0.2 mg/mL. The formulation is provided in a vehicle comprising water having a pH of between about 6.8 and about 7.8 as a solvent. The formulation further comprises sodium chloride, a dibasic sodium phosphate/citric acid buffer, and optionally sodium hydroxide and/or hydrochloric acid to adjust the pH.

The topical bimatoprost formulation may be provided to a patient suffering from skin depigmentation, i.e., vitiligo, in a bottle designed to administer the formulation in a dropwise fashion. The patient may then administer the topical formulation at regular intervals to affected tissue, in an amount of from 1 drop per 5 square centimeters of affected skin to 5 drops per square centimeter of affected skin, preferably 1 drop (where 1 drop is about 0.02 mL to about 0.05 mL, more preferably about 0.03 mL) per 5 square centimeters of affected skin to 1 drop per square centimeter of affected skin, more preferably 1 drop per 2 square centimeters of affected skin. In various embodiments, topical bimatoprost formulations may be administered at intervals ranging from four times per day to once per week, preferably two times per day to twice a week, more preferably two times per day to once a day. Frequency of administration may be adjusted depending on concentration of bimatoprost in the topical formulation, i.e., a topical bimatoprost formulation having a high concentration of bimatoprost may be administered less frequently than a similar formulation having a lower concentration of bimatoprost.

The topical bimatoprost formulation may also be provided to a patient suffering from skin depigmentation, i.e., vitiligo, in a spray bottle designed to administer the formulation as a fine mist or spray. This is useful for patients with large patches of depigmented skin on the trunk, legs, or arms. The patient may administer the formulation as a spray at regular intervals, i.e., daily, twice daily, etc, to affected tissue.

In various embodiments, the method for treating vitiligo in a subject, i.e., a mammal, comprises topically applying a composition comprising bimatoprost to the affected area of skin. For example, one drop of bimatoprost 0.03% solution may be applied for each 2 square centimeters of affected skin. The topical application may be done twice a day without exposing the affected skin to sunlight to accomplish the desired results. The advantage to this treatment regimen is that, unlike psoralen or dermabest gel, exposure to UV lamps or sunlight is not required for the therapy to stimulate melanogenesis. Thus, the risk of sunburn or skin cancer as a result of therapy is lessened.

Topical application of bimatoprost may be used to treat most types of vitiligo. Specifically, topical application of bimatoprost may be used to treat generalized vitiligo (>10% of body surface area involved); Acral or acrofacial vitiligo (vitiligo presenting in a “tip-lip” pattern, affecting the face and distal extremities); localized vitiligo (stable involvement of a small body area) and segmental vitiligo (a single dermatome or extremity is involved—often seen in children). It is unclear at this time if bimatoprost is effective in treating vitiligo affecting the mucosal membranes of the oral or genital areas.

The following examples will serve to illustrate vitiligo treatment by bimatoprost, it being understood that the particulars shown are by way of example and for purpose of illustrative discussion of preferred embodiments of the invention.

### Example 1

### Clinical Efficacy of Bimatoprost for Treatment of Vitiligo

20 Patients with vitiligo were treated with topical bimatoprost solution for 4 months to elucidate its efficacy and tolerability in stable vitiligo. The solution contained bimatoprost in a concentration of 0.3 mg/mL (0.03%), benzalkonium chloride in a concentration of 0.05 mg/mL, and water. The solution has a pH of between 6.8 and 7.8, and contains sodium chloride, a dibasic sodium phosphate/citric acid buffer, and sodium hydroxide and/or hydrochloric acid to adjust the pH.

The number of subjects with their mean age and types of vitiligo are given in Table 1. The subjects had suffered from vitiligo for a period of between 8 and 80 months. The average period of depigmentation for the patients was 26.7 months.

All the patients were examined by an independent observer. The percentage of repigmentation in form of color or size changes and folliculocentric/peripheral repigmentation were observed. No severe adverse reactions were reported during the trial. The degree of repigmentation depending on type of vitiligo is given in Table 2.

TABLE 1
Vitiligo Patients.

| | No. of subjects | Notes |
|---|---|---|
| Males | 12 | Mean Age: 20.8 years |
| Females | 8 | Mean Age: 21.3 years |
| Type of vitiligo | | |
| Vulgaris | 08 | 08 |
| Focal | 06 | 06 |
| Segmental | 04 | 04 |
| Mucosal | 02 | 02 |

TABLE 2
Degree of Repigmentation.

| Type of | | | | | |
|---|---|---|---|---|---|
| vitiligo | Complete | Excellent | Marked | Moderate | Nil/mild |
| Vulgaris | 2 | 2 | 1 | 0 | 3 |
| Focal | 1 | 4 | 1 | 0 | 0 |
| Segmental | 1 | 2 | 1 | 0 | 0 |
| Mucosal | 0 | 0 | 0 | 0 | 2 |

TABLE 3
Repigmentation of Body Sites.

| Sites | Complete | Excellent | Marked | Moderate | Nil/mild |
|---|---|---|---|---|---|
| Head & neck | 4 | 4 | 2 | 0 | 0 |
| Trunk | 0 | 2 | 2 | 0 | 2 |
| Extremities | 0 | 0 | 0 | 2 | 2 |
| Mucosae* | 0 | 0 | 0 | 2 | 2 |

*Data in this row describes repigmentation of mucosal sites in patients having either mucosal vitiligo, or non-mucosal vitiligo which has progressed to include depigmentation of mucosal surfaces.

The grading of repigmentation, as seen in Table 2, is based on the percentage of skin surface which has undergone repigmentation, and/or the change in skin color of depigmented skin. Specifically, Nil/Mild repigmentation means that the skin has undergone less than 25% repigmentation, i.e., less than 25% of the skin surface has repigmented or, if repigmentation is observed, the skin has regained less than 25% of its natural color. Moderate repigmentation means that the skin has undergone 25-49% repigmentation, as measured by repigmented surface are and/or skin color. Marked repigmentation means that the skin has undergone 50-74% repigmentation, and excellent repigmentation means that the skin has undergone 75-99% repigmentation. Complete response means that repigmented skin lesions from vitiligo are indistinguishable from skin which has never been depigmented.

The repigmentation effect of the composition starts rapidly within one to two months after starting the treatment. Topical treatment with bimatoprost is continued for a period of at least two months. If no response is observed to treatment with topical bimatoprost, treatment is stopped at the end of two months. If a mild or moderate response to treatment with topical bimatoprost is observed, therapy is continued for at least 4 months, while observing the patient for any further delayed improvement. In cases where complete or excellent response to topical bimatoprost is observed, treatment is stopped to determine whether relapse, i.e., whether loss of pigmentation is observed upon cessation of bimatoprost therapy. The frequency of application of the topical bimatoprost as an aqueous solution is twice a day.

In instances where complete repigmentation is achieved, color of the skin treated is identical to that of the normal skin areas and the intensity of the color does not increase or decrease rapidly with time. The composition of the invention is effective in lesions caused by vitiligo. Thus the treatment indicates the stability of repigmentation even after withdrawing the therapy.

As seen in Table 2, repigmentation was observed in 14 out of 20, or 70% of patients. The mean onset of pigmentation was 2 months after initiation of therapy with topical bimatoprost. New patients, i.e., patients who had vitiligo for 6 months or less, responded most quickly to therapy with topical bimatoprost, with repigmentation commencing at about four to six weeks after initiation of therapy. Complete clearance of vitiligo lesions, i.e., 100% repigmentation, was seen in 4 out of 20 patients, or 30% of patients. Vitiligo lesions in patients exhibiting complete repigmentation were generally restricted to the face and/or neck. Marked to excellent repigmentation, i.e., 50-99% repigmentation, was seen in 55% of patients.

Further, all patients with segmental or focal vitiligo exhibited at least 50% repigmentation during the trial, i.e., at least marked repigmentation. More than 60% of patients with vitiligo vulgaris, or generalized vitiligo, exhibited at least 50% repigmentation during the trial, i.e., at least marked repigmentation; little or no repigmentation was seen in the remaining patients with generalized vitiligo. Patients with vitiligo restricted to the mucosal surfaces or to the extremities, i.e., arms, legs, or fingers, showed little or no repigmentation. The incidence of side-effects during therapy with topical bimatoprost was 20%, and these side effects were mild. The most commonly observed side effect was a transient burning sensation after application of topical bimatoprost, especially on the lips.

It is observed that the degree of repigmentation depends on the site of application. Vitiligo lesions on the face, trunk and scalp responded to therapy more quickly than lesions on the extremities or mucosal surfaces. Specifically, depigmentation of the face and/or neck due to vitiligo responds well to topical bimatoprost, with all vitiliginous lesions on the face and neck showing at least 50% repigmentation, as seen in Table 3. Vitiliginous lesions on the trunk also respond well to topical bimatoprost, with 66% of lesions on the trunk showing at least 50% repigmentation. Lesions due to vitiligo on mucosal surfaces or on the extremities respond poorly to topical bimatoprost.

The hypothetical mechanism of repigmentation would be of increased melanogenesis in dermal melanocytes together with increased transfer of melanin granules to the basal melanocytes. Patients who showed excellent or complete repigmentation due to topical bimatoprost were followed up for 6 months after stopping bimatoprost treatment, as shown in Table 4. These patients were observed to determine whether a positive response, i.e., at least 25% repigmentation of vitiliginous lesions when compared to pigmentation at the start of the study, was observed. Patients exhibiting a positive response were also observed to determine whether relapse, i.e., loss of pigment during the six month period following cessation of therapy, was observed.

As seen in Table 4, 100% of patients with focal or segmental vitiligo exhibited a positive response 6 months after stopping topical bimatoprost therapy. Of these patients, none showed measurable depigmentation after 6 months. Also as seen in Table 4, four of eight patients (50%) with generalized vitiligo, or vitiligo vulgaris, exhibited a positive response 6 months after stopping topical bimatoprost therapy. Of these four patients, one showed measurable depigmentation after 6 months.

Again referring to Table 4, 100% of patients with vitiliginous lesions on the face or neck exhibited a positive response 6 months after stopping topical bimatoprost therapy. Of these patients, none showed measurable depigmentation after 6 months. Overall, 60% of patients with vitiliginous lesions on the trunk or extremities exhibited a positive response 6 months after stopping topical bimatoprost therapy. Of these patients, 60% showed measurable depigmentation after 6 months.

TABLE 4
Follow-Up Results

| | Total no of | | Relapse/loss |
|---|---|---|---|
| Site or type of vitiligo | patients | Response positive* | of pigment** |
| Face and neck | 10 | 10 (100%) | 1 (10%) |
| Trunk | 6 | 4 (66%) | 2 (50%) |
| Extremities | 4 | 2 (50%) | 1 (50%) |
| Mucosal | 2 | 0 | 0 |
| Vulgaris | 8 | 4 (50%) | 1 (25%) |
| Focal | 6 | 6 (100%) | 0 |
| Segmental | 4 | 4 (100%) | 0 |

*Response positive: Percentage refers to ratio of patients exhibiting at least 25% repigmentation after 6 months to total number of patients.
**Relapse: Percentage refers to percentage of patients exhibiting a positive response who show measurable depigmentation following cessation of topical bimatoprost therapy.

The present invention has clinically demonstrated that bimatoprost has an effect qualitatively and quantitatively superior in the process of repigmentation, compared with the existing products. According to clinical trials performed with topical bimatoprost, bimatoprost has demonstrated encouraging results for the treatment of vitiligo. Its use has shown remarkable effectiveness in repigmentation, i.e., at least 50% repigmentation, in 75% of patients treated. Further, in cases of focal or segmental vitiligo, therapy with topical bimatoprost may be stopped after repigmentation is complete, without relapse or loss of pigment. Relapse may occur after stopping treatment with topical bimatoprost in cases of vitiligo vulgaris, or generalized vitiligo. Patients with vitiligo vulgaris may wish to maintain treatment with topical bimatoprost at a reduced dosage or a reduced frequency to prevent relapse. For example, after completion of repigmentation, the frequency of application of topical bimatoprost may be reduced, i.e., from twice daily to once a day or twice a week as needed, or the concentration of bimatoprost in the topical solution may be reduced, i.e., from 0.3 mg/mL to a concentration of 0.1 mg/mL (0.01%).

### Example 2

### Topical Aqueous Bimatoprost Gel

As an example of a non-ophthalmic bimatoprost formulation, a bimatoprost gel is here described. The gel comprises the following:

bimatoprost in an amount of from about 0.005% to about 0.5% by weight, preferably from about 0.005% to about 0.1% by weight, more preferably from about 0.01% to about 0.05% by weight;

the surfactant lecithin in an amount of from about 0.5% to about 10% by weight;

an acrylic acid polymer or copolymer, such as Carbomer 940, used as a gelling agent in an amount of from about 0.5 to about 5% by weight;

at least one preservative or antioxidant selected from the group consisting of alpha-tocopherol in an amount of from about 0.025 to about 1% by weight, butylated hydroxyanisole in an amount of from about 0.01% to about 0.5% by weight; butylated hydroxytoluene in an amount of from about 0.01% to about 0.5% by weight, and benzalkonium chloride in an amount of from about 0.05% to about 1.0% by weight; and

a solvent system containing a hydrophilic organic solvent, such as dimethyl sulfoxide, in an amount of from about 0.0 to about 2.0% by weight; and the balance water.

### Example 3

### Topical Alcoholic Bimatoprost Gel

As a further example of a non-ophthalmic bimatoprost gel formulation, a bimatoprost gel containing an alcoholic solvent is here described. Due to the increased solubility of bimatoprost in ethanol, as opposed to water, the alcoholic gel may be used to administer an increased dose of bimatoprost. The alcoholic gel comprises the following:

bimatoprost in an amount of from about 0.005% to about 20% by weight;

lecithin in an amount of from about 0.5% to about 10% by weight;

an acrylic acid polymer or copolymer, such as Carbomer 940, in an amount of from about 0.5 to about 5% by weight;

at least one preservative or antioxidant selected from the group consisting of alpha-tocopherol in an amount of from about 0.025 to about 1% by weight, butylated hydroxyanisole in an amount of from about 0.01% to about 0.5% by weight; butylated hydroxytoluene in an amount of from about 0.01% to about 0.5% by weight, and benzalkonium chloride in an amount of from about 0.05% to about 1.0% by weight; and

a solvent system containing a hydrophilic organic solvent, such as dimethyl sulfoxide, in an amount of from about 0.0 to about 2.0% by weight; and the balance ethanol or an ethanol/water mixture.

### Example 4

### Topical Bimatoprost Emulsion

A topical bimatoprost emulsion is now described. Due to the increased solubility of bimatoprost in an oil solvent, as opposed to water, the emulsion may be used to administer an increased dose of bimatoprost, when compared to the ophthalmic formulation used in Example 1. The emulsion comprises the following:

bimatoprost in an amount of from about 0.005% to about 30% by weight, preferably about 0.005% to about 10% by weight, more preferably about 0.01% to about 5% by weight;

the surfactant lecithin in an amount of from about 1.0% to about 15% by weight;

a triglyceride oil solvent, such as soybean oil, in an amount of from about 2.0% to about 50% by weight, preferably about 2.0% to about 20% by weight;

at least one preservative or antioxidant selected from the group consisting of alpha-tocopherol in an amount of from about 0.025 to about 1% by weight, butylated hydroxyanisole in an amount of from about 0.01% to about 0.5% by weight; butylated hydroxytoluene in an amount of from about 0.01% to about 0.5% by weight, and benzalkonium chloride in an amount of from about 0.05% to about 1.0% by weight;

optionally an alkali metal, i.e., sodium, salt of ethylenediamine tetraacetic acid as a chelating agent in an amount of from about 0.05% to about 1.0% by weight; and

balance water.

### Example 5

### Topical Bimatoprost Cream

A topical semisolid cream containing an oil phase having bimatoprost dissolved or dispersed therein and an aqueous phase may be devised. The cream may be prepared as a semisolid oil-in-water emulsion, or as a semisolid water-in-oil emulsion. The oil phase may contain higher fatty acids or esters or amides thereof, higher fatty alcohols or esters or amides thereof; mono-, di-, or triglycerides; hydrocarbon waxes or oils; amide waxes or oils; ester waxes or oils; or a mixture thereof.

A suitable cream may be made as a semisolid emulsion comprising an oil phase dispersed in a water phase. The cream comprises bimatoprost in an amount of from about 0.005% to about 25% by weight of the cream formulation, preferably about 0.005% to about 10% by weight, more preferably about 0.01% to about 5% by weight;

an oil phase in which the bimatoprost is dissolved or dispersed, comprising stearyl alcohol in an amount of from about 10% to about 20% by weight of the formulation; stearic acid in an amount of from about 0.1% to about 5% by weight; glyceryl monostearate in an amount of from about 0.5% to about 10% by weight; 2-octyldodecanol in an amount of from about 1% to about 10% by weight; hydrocarbon and/or ester waxes, such as hydrogenated lanolin and/or squalene, in an amount of from about 1.5% to about 25% by weight;

with the balance of the formulation being an aqueous phase containing water, the thickener polyethylene glycol in an amount of from about 1.0 to about 10% by weight of the formulation, and dimethylsulfoxide in an amount of from about 0.05 to about 1.0% by weight.

The cream may contain as additives at least one preservative or antioxidant selected from the group consisting of alpha-tocopherol in an amount of from about 0.025 to about 1% by weight, butylated hydroxyanisole in an amount of from about 0.01% to about 0.5% by weight; butylated hydroxytoluene in an amount of from about 0.01% to about 0.5% by weight, benzalkonium chloride in an amount of from about 0.05% to about 1.0% by weight; and methyl paraben and/or propyl paraben in an amount of from about 0.1% to about 5.0% by weight.

The cream may further contain a topical anesthetic, such as menthol, in an amount of from about 0.1 to about 1.0% by weight. The anesthetic prevents or reduces any itching or burning sensation felt during use of the bimatoprost cream.

### Example 6

### Topical Bimatoprost Lotion

A topical bimatoprost lotion is now described, where a lotion is a fluid composition containing an oil phase and a water phase. Due to the increased solubility of bimatoprost in an oil solvent, the lotion may be used to administer an increased dose of bimatoprost, when compared to the ophthalmic formulation used in Example 1. The lotion comprises the following:

bimatoprost in an amount of from about 0.01% to about 10% by weight, preferably about 0.01% to about 5% by weight, more preferably about 0.01% to about 1% by weight;

an aqueous phase containing water, 1,3-butylene glycol in an amount of from about 1.0 to about 10% by weight of the formulation, ethanol in an amount of from about 0.5 to about 5.0% by weight, and glycerin in an amount of about 5% by weight;

the polyoxyethylene ether of oleyl alcohol as an oily surfactant in an amount of about 1.0 to about 10.0% by weight of the formulation; and

optionally at least one preservative or antioxidant selected from the group consisting of alpha-tocopherol in an amount of from about 0.025 to about 1% by weight, butylated hydroxyanisole in an amount of from about 0.01% to about 0.5% by weight, butylated hydroxytoluene in an amount of from about 0.01% to about 0.5% by weight, and benzalkonium chloride in an amount of from about 0.05% to about 1.0% by weight.

The lotion may additionally comprise the UV-absorbing agent 2-ethylhexyl p-dimethylaminobenzoate in an amount of from about 0.05 to about 5.0% by weight as a sunscreen, and sodium pyrrolidonecarboxylate in an amount of from about 0.1 to about 10% by weight as a moisturizer.

### Example 7

### Topical Bimatoprost Ointment

A topical bimatoprost ointment is now described, where an ointment is an oil-based composition containing an oil phase and, optionally, a water phase. The ointment comprises bimatoprost in an amount of from about 0.01% to about 50% by weight of the formulation, preferably about 0.01% to about 25% by weight, more preferably about 0.01% to about 10% by weight; dissolved or dispersed within an ointment base. The ointment base comprises:

an oil phase in which the bimatoprost is dissolved or dispersed, prepared by mixing from about 5 to about 25 grams stearic acid, preferably about 15 grams stearic acid; from about 0.5 to about 15 grams white wax, preferably about 1 to about 5 grams white wax, more preferably about 2 grams white wax; and from about 0.5 to about 15 grams beeswax, preferably about 1 to about 10 grams beeswax, more preferably about 5 to about 10 grams beeswax; and optionally

an aqueous phase comprising water or a mixture of water and propylene glycol.

The ointment comprises from about 50% to 100% by weight of the oil phase, preferably from about 70% to 100% by weight of the oil phase, more preferably about 80% by weight of the oil phase; and from 0% to about 50% by weight of the aqueous phase, preferably from 0% to about 30% by weight of the aqueous phase, more preferably about 20% by weight of the aqueous phase. The aqueous phase may be added in sufficient amounts to ensure that the ointment is thick and viscous, without being stiff.

### Example 8

### Topical Bimatoprost Microemulsion

A topical bimatoprost microemulsion is now described, where a microemulsion is a fluid composition containing an oil phase finely dispersed within a water phase. The oil phase is dispersed within the water phase, forming droplets or particles of oil having a particle size of from 0.1 to 5000 nanometers, preferably 1 to 500 nanometers, still more preferably 1 to 100 nanometers, most preferably 1 to 80 nanometers. Due to the increased solubility of bimatoprost in an oil solvent, the microemulsion may be used to administer an increased dose of bimatoprost, when compared to the ophthalmic formulation used in Example 1. The microemulsion comprises the following:

an oil phase containing bimatoprost in an amount of from about 0.01% to about 25% by weight of the formulation, preferably about 0.01% to about 10% by weight, more preferably about 0.01% to about 5% by weight; dissolved or dispersed within a mixture of tri-, di-, and monoglycerides in an amount of from about 1 to about 35% by weight of the formulation, preferably about 5% to about 30% by weight, more preferably about 10% to about 25% by weight; dispersed within

an aqueous phase containing water, ethanol in an amount of from about 1 to about 25.0% by weight of the formulation, 1,3-butylene glycol in an amount of from 0 to about 10% by weight, and glycerin in an amount of 0 to about 5% by weight; and

a nonaqueous emulsifier, such as polyethoxylated caster oil, i.e., Cremaphor®, in an amount of about 5 to about 30% by weight of the formulation.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1) A method of stimulating pigmentation in a depigmented skin region of a patient, comprising treating said depigmented skin region with a topical formulation comprising an effective amount of 17-phenyl-18,19,20-trinor-PGF2α ethyl amide in a dermatologically acceptable carrier,
said depigmented skin region having fewer melanocytes per square centimeter than a nondepigmented skin region of said patient, said depigmented skin region being surrounded by or adjacent to said nondepigmented skin region.

2) The method of claim 1, wherein said depigmented skin region has no melanocytes.

3) The method of claim 1, wherein said topical formulation is a cream, a gel, a lotion, a spray, an ointment, an aqueous solution, a nonaqueous solution, or a transdermal patch.

4) The method of claim 1, wherein the dermatologically acceptable carrier is an oily carrier.

5) The method of claim 1, wherein the dermatologically acceptable carrier is an aqueous carrier.

6) The method of claim 5, wherein the carrier further comprises liposomes or micelles.

7) The method of claim 5, wherein the carrier has a pH of between about 4 and about 10.

8) The method of claim 7, wherein the carrier comprises a buffer.

9) The method of claim 1, wherein the carrier comprises an emulsion, said emulsion comprising:
an oil phase containing bimatoprost in an amount of from about 0.01% to about 25% by weight of the formulation, dissolved or dispersed within an oil- or wax-based formulation; and
an aqueous phase containing water and an optional solvent selected from the group consisting of lower alcohols, lower glycols, and glycerin;
said emulsion being selected from the group consisting of an oil-in-water emulsion and a water-in-oil emulsion.

9) A method of treating a patient suffering from segmental or focal vitiligo, comprising treating a depigmented skin surface of said patient with a topical formulation comprising an effective amount of 17-phenyl-18,19,20-trinor-PGF2α ethyl amide in a dermatologically acceptable carrier, said treating step being carried out at regular intervals until repigmentation of said depigmented skin surface is complete.

10) A method of treating a patient suffering from generalized vitiligo, comprising a first step of treating a depigmented skin surface of said patient with a topical formulation comprising a first amount of 17-phenyl-18,19,20-trinor-PGF2α ethyl amide in a dermatologically acceptable carrier, said first amount being effective to promote repigmentation, wherein said first step is carried out at regular intervals until repigmentation of said depigmented skin surface is complete.

11) The method of claim 10, wherein said method further comprises a second step of treating a depigmented skin surface of said patient with a topical formulation comprising a second amount of 17-phenyl-18,19,20-trinor-PGF2α ethyl amide in a dermatologically acceptable carrier,
said second amount being less than said first amount and effective to maintain pigmentation.

12) A method of treating localized skin depigmentation in a patient, comprising treating depigmented skin of said patient by applying thereto a topical formulation comprising an effective amount of 17-phenyl-18,19,20-trinor-PGF2α ethyl amide in a dermatologically acceptable carrier.

13) The method of claim 12, wherein said patient suffers from vitiligo.

14) The method of claim 12, wherein said patient suffers from a condition selected from the group consisting of generalized vitiligo, acrofacial vitiligo, localized vitiligo, and segmental vitiligo.

15) The method of claim 12, wherein said topical formulation is selected from the group consisting of a cream, a gel, a lotion, a spray, an ointment, an aqueous solution, a nonaqueous solution, and a transdermal patch.

16) The method of claim 12, wherein the dermatologically acceptable carrier is an aqueous carrier.

17) The method of claim 16, wherein the carrier further comprises liposomes or micelles.

18) The method of claim 16, wherein the carrier has a pH of between about 4 and about 10.

19) The method of claim 16, comprising treating said patient with a topical formulation comprising about 0.01% to about 0.03% by weight of 17-phenyl-18,19,20-trinor-PGF2α ethyl amide in an aqueous carrier.

20) The method of claim 12, comprising treating said patient with a topical formulation comprising about 0.01% to about 0.03% by weight of 17-phenyl-18,19,20-trinor-PGF2α ethyl amide in an aqueous carrier, said formulation being administered in an amount of from about 0.02 mL to about 0.05 mL per 2 cm² of skin surface.

21) A method of treating a patient suffering from vitiliginous lesions on the face, the neck, or both the face and the neck, said method comprising:
treating said lesions with a topical formulation comprising a first amount of 17-phenyl-18,19,20-trinor-PGF2α ethyl amide in a dermatologically acceptable carrier, said first amount being effective to promote repigmentation of said lesions;
wherein said treating step is carried out at regular intervals until repigmentation of said lesions is complete.

22) A method of treating localized skin depigmentation in a patient, comprising treating depigmented skin of said patient by applying thereto a topical formulation comprising an effective amount of 17-phenyl-18,19,20-trinor-PGF2α ethyl amide in a dermatologically acceptable carrier;
said method further comprising a step of treating said depigmented skin with a composition selected from the group consisting of pseudocatalase cream, dermabest gel, V-Tar, tacrolimus, pimecrolimus, steroids, psoralen, methoxsalen, trioxsalen, glatiramer acetate, and topical calcineurin inhibitors.
